(19) United States
(12) Patent Application Publication
(10) Pub. No.: US 2019/0029680 A1
(43) Pub. Date: Jan. 31, 2019

(54) SUTURES AND METHODS FOR MAKING SUTURES

(71) Applicant: Central Park Diagnostics, Inc., Bayonne, NJ (US)

(72) Inventor: Shahnoz Rustamova, Bayonne, NJ (US)

(21) Appl. No.: 16/149,081

(22) Filed: Oct. 1, 2018

Related U.S. Application Data
(63) Continuation-in-part of application No. 15/082,579, filed on Mar. 28, 2016.
(60) Provisional application No. 62/565,559, filed on Sep. 29, 2017.

(51) Int. Cl. A61B 17/06; A61L 17/00; A61L 17/08

(52) U.S. Cl. CPC A61B 17/06166; A61L 17/08; A61L 17/005

(57) ABSTRACT

Various embodiments of a suture are provided. In one embodiment, a suture comprises a plurality of nanoparticles configured to form a fiber structure.

### CROSS-REFERENCE TO RELATED APPLICATIONS

This application claims the benefit of priority to U.S. Provisional Patent Application No. 62/565,559, “Sutures and Methods for Making Sutures,” filed Sep. 29, 2017, which is incorporated by reference herein.

This application is a continuation-in-part application of U.S. patent application Ser. No. 15/082,579, “SUTURE DELIVERY DEVICE, AND SUTURE, FOR FACILITATING FIBROSIS AND HEALING,” filed Sep. 29, 2016, which is incorporated by reference herein.

### FIELD OF THE INVENTION

Embodiments described in this disclosure relate generally to sutures and related devices, and particularly sutures configured to release drugs and other ingredients.

### BACKGROUND

Many types of sutures, including absorbable sutures, are known. Sutures comprising delivery systems for drugs and other types of agents are also known to promote accelerated healing of a wound or tissue, to provide antibacterial, anti-inflammatory, and/or analgesic properties, and to reduce adhesion and scar formation. See, e.g., U.S. Patent Application Publication No. 20160278770, “SUTURE DELIVERY DEVICE, AND SUTURE, FOR FACILITATING FIBROSIS AND HEALING,” Sep. 29, 2016, incorporated by reference herein. Such delivery systems may comprise, for example, nanoparticles or microparticles of one or more suitable agents configured as a coating for a suture.

### BRIEF DESCRIPTION OF THE DRAWINGS

An understanding of embodiments described in this disclosure and many of the related advantages may be readily obtained by reference to the following detailed description when considered with the accompanying drawings, of which:

FIG. 1 is a diagram of a portion of a suture comprising multiple particle structures according to an embodiment of the present invention;

FIG. 2A is a diagram of a particle structure according to an embodiment of the present invention; and

FIG. 2B is a diagram of a particle structure according to an embodiment of the present invention; and

FIG. 2C is a diagram of a particle structure according to an embodiment of the present invention.

### DETAILED DESCRIPTION

Some embodiments of the present invention provide for a suture promoting accelerated healing of a wound or tissue; to provide antibacterial, anti-inflammatory, and/or analgesic properties; and/or to reduce adhesion and scar formation. In one embodiment, the suture is absorbable.

In some embodiments, a suture may comprise a targeted agent delivery system configured to deliver one or more types of drugs, medicines, minerals, vitamins, other types of supplements, and/or plasma (e.g., collectively referred to as “agents” in this disclosure).

The targeted agent included in the delivery system may be selected, for example, to promote collagen formation (e.g., zinc (Zn), copper (Cu), vitamin C, vitamin A, vitamin D, vitamin B), provide an antibacterial effect, and/or promote regeneration of tissue (e.g., plasma and/or zinc to promote healing of organ tissue). Some examples of agents that may be useful for accelerating and/or promoting healing at the site of a wound or surgical incision include, but are not limited to: biocidal agents, antimicrobial agents, antibiotics, anti-proliferatives, medicants, growth factors, anti-clotting agents, plasma, clotting agents, analgesics, anesthetics, anti-inflammatory agents, wound repair agents and the like, chemotherapeutics, biologics, protein therapeutics, monoclonal or polyclonal antibodies, DNA, RNA, peptides, polysaccharides, lectins, lipids, probiotics, diagnostic agents, angiogenics, anti-angiogenic drugs, polymeric drugs, epigenic enzymes (e.g., histone deacetylase (HDAC1, HDAC3, HDAC8), P21, P53 (apoptosis), CaCl, CA gluconate, iron oxide, vitamin C, diteruloymethane (curcumin), demethoxycurcumin (DMC), bisdemethoxycurcumin (BDMC), and combinations thereof.

In one or more embodiments, the targeted delivery system may comprise one or more nanostructures, such as a nanoparticle (NP) or carbon nanotube (CNT). Various types of NP structures and/or coatings are known to those skilled in the art. In some embodiments, the NPs may be loaded with one or more agents (some examples of which are discussed above), and targeted to the area of the body in which the suture is placed, allowing the delivered agent to affect the local tissue (e.g., injured tissue and/or tissue in which to promote fibrosis). In this way, one or more types of agents may be inserted into the body and targeted locally, avoiding interaction with other parts of the body and/or with other (healthy) tissue.

As will be understood by those skilled in the art, NPs may contain encapsulated, dispersed, absorbed, or conjugated drugs, supplements, and/or other types of agents. As will be understood by those skilled in the art, NPs may include colloidal drug delivery systems comprising particles with a size range from 10 to 1000 nm in diameter, but other alternatives (e.g., particles larger than 1000 nanometers in size) for delivery of agents may be desirable for some implementations.

In one example, NPs may be coated by a polymer, which releases the coated agent(s) by controlled diffusion, or erosion from the core across the polymeric membrane or particle matrix. Accordingly, the solubility and diffusivity of an agent in the polymer membrane determines the release rate of the agent.

NP size and surface characteristics may be configured to achieve both passive and active agent targeting. Site-specific targeting can be achieved by attaching targeting ligands, such as antibodies or aptamers, to the surface of NPs, or by using guidance in the form of magnetic NPs. NPs may also control and sustain release of an agent during transport to, or at, the site of localization, altering agent distribution and subsequent clearance of the agent in order to improve therapeutic efficacy and reduce side effects.

Various nanoforms have been attempted as agent delivery systems, varying from biological substances, such as albumin, gelatin and phospholipids for liposomes, to chemical substances, such as various polymers and solid metal-containing NPs. In one embodiment, a suture may comprise a polymer-drug conjugate, which may have a high size variation normally not considered as an NP. As noted above, nanodelivery systems may be designed to have agents absorbed or conjugated onto the particle surface, encapsulated inside the polymer/lipid or dissolved within a particle matrix.

According to some embodiments, a nanoparticle, or other type of targeted agent delivery system, may comprise one or more types of agents. In one example, a nanoparticle (e.g., attached to a suture) may comprise two or more types of agents, such as a first agent that promotes a fibrotic effect in tissue and a second agent that promotes a regenerative effect in tissue (e.g., to improve a rate of healing of a wound).

In accordance with some embodiments, a suture (referred to in this disclosure as a “nano suture”) comprises at least one targeted agent delivery system comprising at least one nanoparticle. In one example, a nano suture comprises a nanoparticle comprising an agent promoting a fibrotic effect in tissue. In another example, a nano suture comprises a nanoparticle comprising an agent promoting regeneration in tissue (e.g., to increase the rate of healing of a wound in a uterine wall).

In accordance with some embodiments, a nano suture may comprise any number of nanoparticles. In some embodiments, a nano suture may comprise any number of different types of nanoparticles, which may differ with respect to structure, coating, and/or agent(s)).

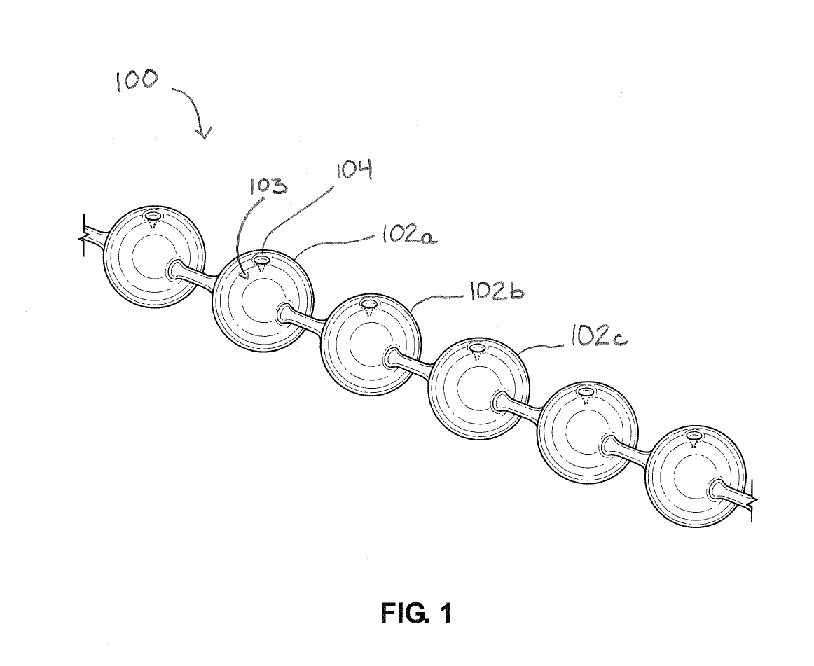

In accordance with some embodiments, a nano suture may comprise a plurality of nanoparticles connected to one another, such as in a series, matrix, or any other desirable configuration. In one embodiment, as shown in FIG. 1A, a nano suture may comprise a plurality of nano or micro particles connected to one another in a string or fiber configuration.

In accordance with some embodiments, a plurality of nano sutures may be combined and/or adhered to one another to form a larger suture structure with an appropriate rigidity and flexibility for a desired medical purpose.

In some embodiments, a plurality of nano sutures may be encased, for example, in an outer casing of nano or micro particles and/or in an outer casing of absorbable suture material (e.g., polymer) in order to provide structure to the plurality of nano sutures.

According to some embodiments, a nano suture may be constructed of multiple beads or other nanostructures containing agents that are released upon direct contact with tissue/plasma, wherein the agents are encapsulated in expandable polytetrafluorethylene (ePTFE) or collagen.

According to one embodiment, the beads that make up a nano suture may comprise a one-way system that allows plasma to flow inward freely (e.g., due to osmosis), where its activation takes place by all ingredients of the bead. In another example, a bead may comprise a one-way valve-opening system that permits flow of plasma only into the bead.

In some embodiments, each bead of a nano suture is configured to burst after complete or substantially complete saturation of the plasma, and thus all activated ingredients are released, providing the desired effect on the tissue according to the properties of the ingredient(s).

In accordance with some embodiments, different ingredients and components of a suture, and their respective effects on tissue, may provide for: natural glue (iron oxide), pain (DMC, BDMC, curcumin), antibacterial (DMC, BDMC, curcumin), and prevention of adhesion formation (e.g., EPTFE).

In accordance with some embodiments, an extrusion, solvent, and/or molding process may be utilized to produce one or more types of sutures described in this disclosure. Plastic molding methods, for example, are known to those skilled in the art and include, but are not limited to, melt molding (e.g., injection molding, extrusion molding, and/or compression molding) and solution molding.

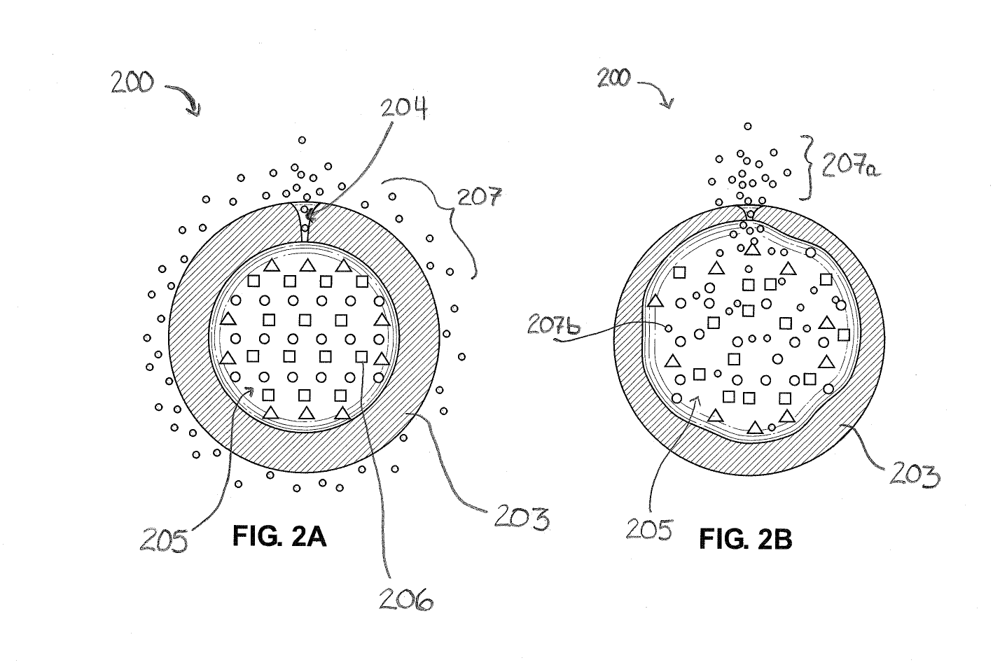

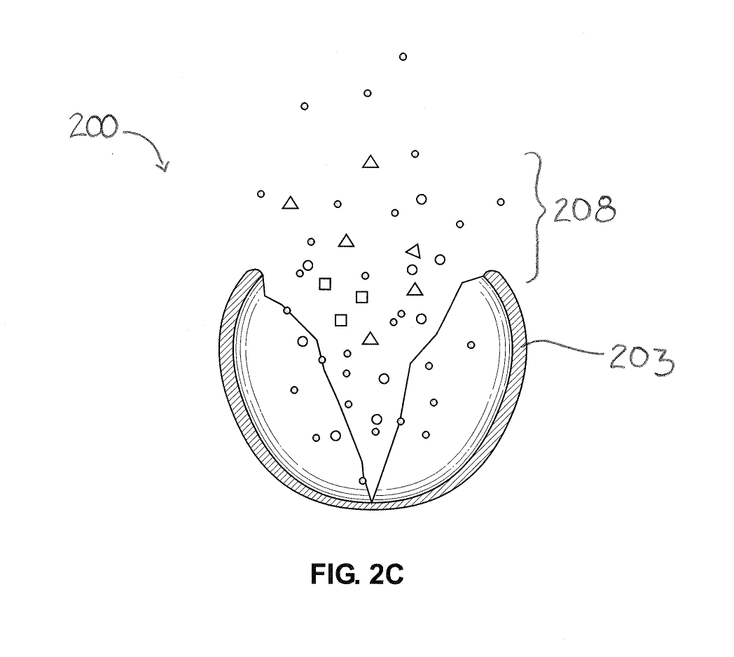

The beads or nano particles are not necessarily represented to scale in FIGS. 1, 2A, 2B, and 2C. In accordance with some embodiments, as described in this disclosure, a bead may represent a targeted delivery system comprising one or more nanoparticles, including one or more types of agents (e.g., an agent for producing a fibrotic effect and/or a second agent for accelerating healing or tissue regeneration).

Additional disclosure, in accordance with some embodiments, is provided in FIGS. 1A-C. FIG. 1 shows a string of nano or micro particles 102a-c (referred to as “beads” in this disclosure) forming a nano or micro suture fiber 100. One or more beads 102a-c of the suture fiber 100 may comprise an outer shell 103 comprising a one-way valve 104. Such structures may be formed, for example, of natural collagen or ePTFE, and may include, as discussed in this disclosure, one or more types of agents.

According to some embodiments, a suture may be formed using a plurality of suture fibers such as the example depicted in FIG. 1. For example, a suture's “core” may be formed of a plurality of suture fibers (e.g., suture fiber 100) comprising beads of ePTFE and/or collagen.

In some embodiments, as depicted in FIGS. 2A, 2B, and 2C, a bead 200 may comprise a one-way valve opening system 204 in shell 203 that, once the suture of which bead 200 is a part is implanted, allows plasma 207 (e.g., in the body) to flow in through the valve 204. The valve 204 is configured so that any fluids and agents 206 in the interior chamber 205 of the bead 200 cannot escape. Although three different types of agents are depicted in FIG. 2A (by use of three different symbol types), it will be readily understood in light of this disclosure that any number and type of agents 206 may be used as desired for a particular implementation and function.

As depicted in FIG. 2B, some of the plasma 207b enters the interior chamber 205, and as additional plasma 207a continues to enter the interior chamber 205 (e.g., via osmosis), the chamber fills up with plasma, the shell 203 deforms and/or expands. Eventually, as depicted in FIG. 2C, the volume of plasma 207b in interior chamber 205 of the shell 203 bursts the bead 200, releasing contents 208 of the bead 200, including the agents 206 (e.g., CaCl, vitamin C, iron oxide, curcumin, etc.), which may be activated on release.

The dimensions of a suture body (e.g., comprising a plurality of nano sutures), in accordance with some embodiments, may be of any size suitable for a desired surgical use. According to some embodiments, the diameter of the body of a suture may be configured specifically for use in a cosmetic surgery procedure (or one or more specific types of cosmetic surgery). In one embodiment, a diameter and/or length of the thread body may be configured for use in a face lift or other type of rejuvenative surgery procedure, which may require a diameter and/or length relatively smaller than for a suture used in cosmetic surgery involving other portions of the body (e.g., the abdomen).

Various methods are known for the manufacture of nano particles. Examples that may be suitable for creating nano particles described in this disclosure include but are not limited to vapor condensation, chemical synthesis, and attrition. Manufacture parameters such as temperature, time, and reaction phase are known to affect the size of the resultant nano particles an may be manipulated to provide nano particles of a desired size.

### Interpretation

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention may be practiced with various modifications and alterations, such as structural, logical, software, and/or electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments nor a listing of features that must be present in all embodiments.

Neither the Title (set forth at the beginning of the first page of this disclosure) nor the Abstract (set forth at the end of this disclosure) is to be taken as limiting in any way the scope of the disclosed invention(s).

Throughout the description and unless otherwise specified, the following terms may include and/or encompass the example meanings provided below. These terms and illustrative example meanings are provided to clarify the language selected to describe embodiments both in the specification and in the appended claims, and accordingly, are not intended to be limiting.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise.

The terms “the invention” and “the present invention” and the like mean “one or more embodiments of the present invention.”

A reference to “another embodiment” in describing an embodiment does not imply that the referenced embodiment is mutually exclusive with another embodiment (e.g., an embodiment described before the referenced embodiment), unless expressly specified otherwise.

The terms “including”, “comprising” and variations thereof mean “including but not limited to”, unless expressly specified otherwise.

The terms “a”, “an” and “the” mean “one or more”, unless expressly specified otherwise.

The term “plurality” means “two or more”, unless expressly specified otherwise.

The term “herein” means “in the present disclosure, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

Each process (whether called a method, algorithm or otherwise) inherently includes one or more steps, and therefore all references to a “step” or “steps” of a process have an inherent antecedent basis in the mere recitation of the term “process” or a like term. Accordingly, any reference in a claim to a “step” or “steps” of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

As used in this disclosure, a “user” may generally refer to any individual and/or entity that operates a device.

A description of an embodiment with several components or features does not imply that any particular one of such components and/or features is required. On the contrary, a variety of optional components are described to illustrate the wide variety of possible embodiments of the present invention(s). Unless otherwise specified explicitly, no component and/or feature is essential or required.

Further, although process steps, algorithms or the like may be described or depicted in a sequential order, such processes may be configured to work in one or more different orders. In other words, any sequence or order of steps that may be explicitly described or depicted does not necessarily indicate a requirement that the steps be performed in that order. The steps of processes described in this disclosure may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications, does not imply that the illustrated process or any of its steps is necessary to the invention, and does not imply that the illustrated process is preferred.

While the present invention has been illustrated by a description of various embodiments and while these embodiments have been described in considerable detail, it is not the intention to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the general inventive concept.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicant reserves the right to file additional applications to pursue patents for subject matter that has been disclosed and enabled but not claimed in the present application.

What is claimed is:

1. A suture comprising:
a plurality of suture fibers,
each suture fiber comprising a plurality of connected nano structures,
each nano structure comprising at least one ingredient contained in a shell, the shell allowing for one-way flow of plasma into the shell for activating the at least one type of ingredient.

2. The suture of claim 1, wherein the at least one type of ingredient promotes the production of fibrotic tissue.

3. The suture of claim 1, wherein the at least one type of ingredient comprises a chemical agent.

4. The suture of claim 1, wherein the at least one type of ingredient comprises a biological agent.

5. The suture of claim 1, wherein the at least one type of ingredient comprises a hormonal agent.

6. The suture of claim 1, wherein the shell is configured to allow plasma to enter the shell.

7. The suture of claim 1, wherein the shell comprises a one-way valve opening system configured to allow plasma to enter the shell.
